(19) United States
(12) Patent Application Publication
(10) Pub. No.: US 2009/0068440 A1
(43) Pub. Date: Mar. 12, 2009

(54) PRODUCTION OF ACROLEIN, ACRYLIC ACID AND WATER-ABSORBENT POLYMER STRUCTURES MADE FROM GLYCERINE

(76) Inventors: Gunther Bub, Marl (DE); Jurgen Mosler, Castrop-Rauxel (DE); Andreas Sabbagh, Dulmen (DE); Franz-Felix Kuppinger, Marl (DE); Guido Stochniol, Haltern (DE); Jorg Sauer, Dulmen (DE); Jorg Leistner, Dortmund (DE); Gunter Latoschinski, Marl (DE); Thorsten Schwatzke, Marl (DE)

Correspondence Address: SMITH MOORE LEATHERWOOD LLP, P.O. BOX 21927, GREENSBORO, NC, 27420, US

(21) Appl. No.: 11/917,850

(22) PCT Filed: Jun. 16, 2005
(86) PCT No.: PCT/EP06/05793

§ 371 Date: Aug. 15, 2008

(30) Foreign Application Priority Data
Jun. 20, 2005 (DE) 10 2005 028 624.0

(51) Int. Cl. B32B 5/16; C07C 47/02; C07C 51/235; B01J 19/24; C08F 20/06; B32B 27/30

(52) U.S. Cl. 428/327; 568/448; 562/532; 422/188; 526/317.1; 428/500

(57) ABSTRACT

The present invention relates to a process for the production of acrolein, comprising the following steps:
(a) bringing into contact of an aqueous glycerine phase in an acrolein reaction area to obtain an aqueous acrolein reaction phase;
(b) depleting the acrolein from the acrolein reaction phase to obtain an acrolein phase and a depleted acrolein reaction phase;
(c) conducting back at least a part of the depleted acrolein reaction phase into the acrolein reaction area.
   The invention further relates to a process for production of acrylic acid as well as of water-absorbing polymer structures, composites, in particular hygiene articles, comprising these water-absorbing polymer structures, a process for production of the composites and further chemical products based on the acrylic acid obtained by the inventive process and also the use of this acrylic acid in chemical products.

This application is a national stage application under 35 U.S.C. 371 of international application No. PCT/EP2006/005793 filed 16 Jun. 2006, and claims priority to German Application No. DE 10 2005 028 624.0 filed 20 Jun. 2005, the disclosures of which are expressly incorporated herein by reference.

### BACKGROUND

The invention relates to a process for production of acrolein, acrylic acid and of water-absorbing polymer structures, and composites, in particular hygiene articles, comprising these water-absorbing polymer structures, a process for production of these composites, as well as further chemical products based on the acrylic acid obtained by the process according to the invention and also the use of this acrylic acid in chemical products.

In GB 141 057 a process for dehydration of glycerine to form acrolein is described, in which the reaction is carried out at about 200° C. at a mixture of potassium hydrogensulfate and potassium sulphate. This process leads, however, to only unsatisfactory selectivities, which, in addition, significantly decrease in the course of a longer reaction. Thus, this process is poorly suited to industrial use. By selectivity is understood the quotient of the molar amount of generated product and the molar amount of a reference component, here, glycerine. For continuously operated systems, the quotient of the molar flow is considered.

Furthermore, FR 695 931 describes another method for dehydration of glycerine to acrolein at a solid state catalyst. From the repeat of this process carried out in DE 42 38 493, it may be seen that the yields of this process are not sufficient for technical use.

In DE 42 38 493, both gas phase and liquid phase reactions at a solid state catalyst for conversion of glycerine to acrolein are described. With high selectivities, only comparably low turnovers were achieved, which, in addition, decrease with increasing turnover.

Although this process is interesting for an industrial use in view of the high selectivities, the turnovers achieved and the reduction of selectivity are in need of improvement.

In WO 03/051809, a process for production of acrylic acid starting from propylene via acrolein is disclosed, which is perfectly suited for industrial production of acrylic acid. Besides propylene, which is generally obtained from petrochemical processes, such as naphtha cracking, there exits, however, a further route to the production of acrylic acid, which is not based on a petrochemical but on native (renewable) raw materials, via glycerine, which is produced, for example, by fat saponification, fat splitting, as well as during biodiesel production.

The object of the present invention is first, generally to alleviate or even to overcome the disadvantages arising from the state of the art.

A further object of the present invention is to provide a process for production of acrolein from glycerine, which is suitable for industrial use and, in particular, has satisfactory turnover and selectivities.

A further object according to the invention is to provide a process for production of acrolein, which generates an acrolein phase, which is suitable for feeding into the further step, namely the conversion of acrolein to acrylic acid by oxidation.

In addition, an object according to the invention is to provide a process for production of acrylic acid which may find industrial application. Furthermore, polyacrylates, in particular water-absorbing polyacrylates, also called superabsorbers, are used in many applications, so that it is a general requirement to produce these polyacrylates at least partially on the basis of renewable raw materials and thus to provide polyacrylates based at least partially on renewable raw materials. This is of particular interest in particular for water-absorbing polymers, since the water-absorbing polymers produced to date based on renewable raw materials, for example from celluloses, have significantly worse absorption and water-retention properties than the water-absorbing polymers based on polyacrylates. This has, in turn, a disadvantageous effect on composites comprising these water-absorbing polymers, in particular hygiene articles. These become as a rule more voluminous, which leads to a larger waste volume and worsened wearer comfort, and, in addition, have worse water-retention properties and more leakage.

Thus, a further object according to the invention consists in helping to alleviate the disadvantages described in the above paragraph or even to overcome them.

Furthermore, an object according to the invention consists in providing polyacrylates and in particular water-absorbing polymers which are gentler on resources, which are not inferior in their physical properties to previous polyacrylates and in particular water-absorbing polymers.

Furthermore, an object of the present invention is to provide composites, in particular hygiene articles, which are acceptable from an ecological point of view, which are not inferior in their properties to previous composites and in particular hygiene articles.

A contribution to the solution of at least one of the above objects is provided by the subject matters of the category-forming independent principal and adjacent claims, whereby the therefrom dependent sub-claims represent preferred embodiments of the present invention, whose subject matters likewise make a contribution to solving at least one object.

### SUMMARY

According to an embodiment, the invention relates to a process for production of acrolein, at least comprising the following steps:
(a) bringing an aqueous glycerine phase into an acrolein reaction area to obtain an aqueous acrolein reaction phase;
(b) depleting the acrolein from the acrolein reaction phase to obtain an acrolein phase and a depleted acrolein reaction phase; and
(c) conducting back at least a part of the depleted acrolein reaction phase into the acrolein reaction area.

According to another embodiment, the invention relates to a process for production of acrylic acid, comprising at least the following steps:
(A) bringing an aqueous glycerine phase into an acrolein reaction area to obtain an aqueous acrolein reaction phase;
(B) depleting the acrolein from the acrolein reaction phase to obtain an acrolein phase and a depleted acrolein reaction phase;
(C) conducting back at least a part of the depleted acrolein reaction phase into the acrolein reaction area; and
(D) oxidation of the acrolein from the acrolein phase to acrylic acid in the gas phase at a gas phase catalyst.

### FIGURE

The foregoing and other features, aspects, and advantages of the present invention will become better understood with regard to the following description, appended claims, and accompanying drawing where:

FIG. 1 shows schematically, a first embodiment of an arrangement according to the invention.

### DETAILED DESCRIPTION

In general, in the conducting back, the back-conducted flow is adjusted so that high acrolein yields may be obtained with turnovers which are as high as possible. The return ratio of the glycerine phase to the conducted back depleted acrolein reaction phase may lie within the range from about 0.01:10 to about 9:10, or from about 0.1:10 to about 5:10, or from about 0.5:10 to about 3:10. The conducting back serves to protect the environment. For the case that no back-conducting occurs, the depleted acrolein reaction phase must be removed in some other way. This may occur by dumping, in purification plants, or in combustion plants. Accordingly, the process according to the invention is also possible without recycling, which may not be advantageous for environmental reasons.

The acrolein reaction phase in the acrolein reaction area may have a pressure of at least about 50, or at least about 80, or at least about 120, or at least about 140 bar. The acrolein reaction area is thus designed as a pressure area, which is limited at its start by a pressure generator such as a pump and at its end by a pressure regulator, such as a pressure valve. The dehydration reaction may occur at least in a part of the acrolein reaction area. Generally, the acrolein reaction area may be at least partially formed like a pipe, and designed for up to a maximum pressure load of about 500 bar and a maximum temperature load of about 600° C., which are sufficient for carrying out the process according to the invention.

In addition, the acrolein reaction phase in the acrolein reaction area may have a temperature of at least about 80° C., or at least about 180° C., or at least about 230° C., or at least about 280° C., or at least about 320° C. The temperatures may, on the one hand, be achieved via the pressure ratios in the acrolein reaction area as well as via a corresponding heating of the acrolein reaction phase. In general, the pressure and/or temperature conditions in the acrolein reaction phase in the acrolein reaction area may be selected so that the acrolein reaction phase and in particular the water comprised therein are at least close to or at least partially in the supercritical region.

The glycerine phase may comprise less than about 10 wt %, or less than about 8 wt %, or less than about 6 wt % glycerine, based on the total weight of the glycerine phase, whereby the minimum amount of glycerine in the glycerine phase is about 0.01 wt %, or about 0.1 wt. % or about 1 wt. %.

In addition, the acrolein reaction area may comprise, besides water, a dehydration catalyst. This may be present in an amount from about 0.001:1000 to about 10:1000, or from about 0.01:1000 to about 5:1000 or from about 0.04:1000 to about 1:1000, respectively based upon the amount of glycerine used in the acrolein reaction phase.

The dehydration catalyst may be present either as an acid or as a base or as a combination thereof. If the dehydration catalyst is present as acid, this acid may be a compound, besides water, which also acts as a strong acid close to or within the supercritical region, which has acidic properties. If the dehydration catalyst is an acid, both inorganic and organic acids may be considered. Inorganic acids may include phosphorus acids such as H₃PO₄, sulphur acids such as H₂SO₄, boron acids such as B(OH)₃, or a mixture thereof. In a further embodiment of the dehydration catalyst, this is present as a superacid, which, according to the definition, has a small pK_{S} value of <−1. If the dehydration catalyst is present as an organic acid, alkylsulfonic acids may be used, whereby trifluoromethanesulfonic acid or methanesulfonic acid or mixtures thereof are examples. As bases are considered in connection with the dehydration catalyst, examples include aluminum, lanthanum, alkali or alkaline earth oxides, hydroxides, phosphates, pyrophosphates, hydrogen phosphates or carbonates, or a mixture of at least two thereof, which may be respectively also be on a carrier.

Furthermore, the dehydration catalyst may be present at room temperature both as a solid as well as a liquid. Fluid dehydration catalysts immobilized on a solid carrier also fall under the dehydration catalysts present as solid. Solid dehydration catalysts may include silicon oxide-comprising compounds such as zeolites. In addition, Ti, Zr, or Ce oxides, sulfatized oxides, and phosphatised oxides, or mixtures of at least two thereof are also considered.

A number of dehydration catalysts is described more closely in DE 42 38 493.

The acrolein reaction phase may comprise a fluid different from water. For the case when fluid dehydration catalysts are used, this fluid should also be different from these catalysts. These fluids have a function as solubility improvers. In general, organic compounds that are water-miscible at about 20° C. may be considered as such fluids, which comprise at least one hetero-atom, or two hetero-atoms, and may be inert with respect to other components of the acrolein reaction phase. Such fluids may include, for example, hydroxypiperidine, or aprotic, and polar fluids such as sulfolane, diglyme, tetraglyme, dioxane, trioxane, or γ-butyrolactone. Furthermore, compounds are considered as fluids that have a chelating effect.

In this context, EDTA, NTA, or DPTA are examples, as obtainable under the trade names Versene®, Versenex®, Entarex®, or Detarex®, or also crown ethers.

In another embodiment, the acrolein reaction area may comprise a metal, or a metal compound, or both. This compound may be a mono-, di-, or multivalent metal, or metal compounds. This metal or these metal compounds may be different from the metal or metals which are used in the construction of the acrolein reaction area. This also corresponds to an embodiment according to the invention that these metals or metal compounds are immobilized directly or indirectly with assistance of an adhesive agent to the material used for the construction of the acrolein reaction area. These metals or metal compounds may, however, also be present in particulate form in the acrolein reaction area. These metals or metal compounds should not be carried out of the acrolein reaction area by a fluid or gas flow. This may be achieved, in addition to the immobilization of these metals or metal compounds in the case where they are present in particulate form, by suitable sieves or filters provided in the acrolein reaction area. Furthermore, it further corresponds to an embodiment of the process according to the invention that the metals or metal compounds respectively may be selected so that the above-mentioned fluids may coordinate or complex to these metals or metal compounds. In addition, these metals may be present as metal compounds, whereby metal salts or metals complexed with ligands are examples. As ligands are considered in particular carbon monoxide such as carbonyl, triphenylphosphine, Cp, Cp*, or AcAc are examples. The metal salts may be used in particular in the form of their sulphates or phosphates. Metals may include tin, such as tin sulphate, zinc such as zinc sulphate, lithium such as lithium sulphate, magnesium such as magnesium sulphate, copper such as copper sulphate, palladium such as palladium carbonyl complex, which is mostly used as acetate, rhodium such as rhodium carbonyl complex, which is mostly used as acetate, ruthenium such as ruthenium carbonyl complex, which is mostly used as acetate, nickel such as nickel carbonyl complex, which is mostly used as acetate, iron such as iron carbonyl complex, cobalt such as cobalt carbonyl complex, caesium such as caesium acetate as well as lanthanides, lanthanum, or a mixture of at least two thereof. The metals may be used as salts with complexing agents, often also in the presence of carbon monoxide. Heteropolyacids are examples of metal compounds. Examples of heteropolyacids include those that arise if different types of acidic molecules of a metal such as of chromium, tungsten, or molybdenum, and a non-metal, such as phosphorus, come together with discharge of water. Heteropolyacids may include for example, phosphorus-tungsten acids, silico-tungsten acids, or silico-molybdenum acids, and also the corresponding vanadium compounds.

The dwell time of the acrolein reaction phase in the acrolein reaction area may lie from about 1 to about 10,000 seconds, or from about 5 to about 1,000 seconds, or from about 10 to about 500 seconds.

In addition, it has been shown to be helpful that the acrolein reaction phase comprises carbon monoxide from about 0.0001 to about 10 wt %, or from about 0.001 to about 7 wt %, or from about 0.005 to about 5 wt %, respectively based upon the acrolein reaction phase. This measure may be advantageous for the reduction of side-components.

In addition, the acrolein reaction phase at the end of the acrolein reaction area may comprise an amount of less than about 50 wt % glycerine, or less than about 25 wt % glycerine, or less than about 15 wt % glycerine, and an amount of from about 0.1 to about 50 wt. %, or of from about 1 to about 40 wt %, or from about 5 to about 30 wt % of acrolein, respectively based upon the acrolein reaction phase. By this way of carrying out the process, an acrolein phase may be obtained that may be fed into step (D) over a substantially longer time, without notable worsening of the conversion of acrolein to acrylic acid. It is, furthermore, generally the case in the process according to the invention that the glycerine concentration at the start of the acrolein reaction area is greater than at the end of the acrolein reaction area and may continuously reduce towards the end.

According to a particular embodiment of the process of the invention, the turnover in the acrolein reaction area is at least about 25%, or at least about 26%, or at least about 30%, or at least about 50%. A turnover of at least about 25% means here that at least about 25% of the glycerine molecules entering the acrolein reaction area are converted into acrolein.

At least a part of the acrolein reaction phase may be present in gaseous form. The acrolein reaction phase in the acrolein reaction area may be present in at least two aggregate states. These aggregate states may be liquid and gaseous. For the case that at least a part of the acrolein reaction phase is present as a gas, the concentration in acrolein in this acrolein reaction gas phase may be higher than in the part of the acrolein reaction phase that has a different aggregate state to the acrolein reaction gas phase. A depletion or respective separation of the acrolein is possible considerably more simply by means of the high acrolein concentration in the acrolein reaction gas phase, in that predominantly the acrolein reaction phase from the acrolein reaction area, which is highly concentrated in acrolein, may be discharged by a corresponding pressure regulation, and then acrolein may be obtained in high concentration by releasing pressure.

The purer the thus-obtained acrolein, the less it is necessary that, in addition to the release of pressure, which may occur, for example, by means of a pressure regulator formed as a pressure regulating valve, a cooling by means of a heat exchanger and a further separation, which generally occurs distillatively, a separating unit is necessary. It is further possible that the acrolein reaction phase leaving the acrolein reaction area may be conducted via a plurality of units connected one after the other and consisting of an over-current valve and a heat exchanger, before the thus-created acrolein phase is conducted to a separating unit. The pressure difference before the pressure regulator in the acrolein reaction area, and after the pressure regulator, is preferably at least about 30 bar, or at least about 60 bar, or at least about 100 bar. The acrolein in the acrolein reaction area may at least partially be present in a supercritical state, which contributes to the increased yield.

The acrolein concentration in the acrolein reaction phase before the depletion may be higher by at least about 5%, or at least about 10%, or at least about 50% than after the depletion. A carrier gas may be used in the process. This carrier gas may be supplied before the acrolein reaction area and serves to discharge the acrolein reaction phase. Also in this context, it is advantageous to find as much acrolein as possible in a gaseous part of the acrolein reaction phase. As carrier gas, in principal, all gases that are inert with respect to the compounds participating in the above process may be considered. Examples for carrier gases of this type include but are not limited to nitrogen, air, CO₂, water, or argon. The carrier gas may at least be partially fed back into the acrolein reaction area after passing through the acrolein reaction area. This feed may occur directly before the acrolein reaction area or also at any other position before the acrolein reaction area and may be used in order to form a pre-pressure of the reactants, which are further compressed by means of a corresponding pump to the pressure conditions necessary for the acrolein reaction area.

In the process according to the invention for production of acrylic acid, the acrolein phase in step (D) may comprise acrolein of from about 5 to about 30 wt %, or from about 7 to about 20 wt %, or from about 10 to about 20 wt %, respectively based on the acrolein phase. In connection with as long a life as possible for the oxidation reactor in step (D), the acrolein phase may comprise less than about 10 wt %, or less than about 5 wt %, or less than about 2 wt % components which are generally described as high-boilers, and may have a higher boiling point than acrolein. The acrolein phase may comprise less than about 10 wt %, or less than about 5 wt %, or less than about 2 wt. %, respectively based on the acrolein phase, of low-boilers, i.e. materials which have a lower boiling point than acrolein. In another embodiment, the acrolein phase, in addition to acrolein and optionally present low- or high-boilers, respectively, may comprise substantially inert components, in particular gaseous components, which only negatively affect the oxidation reaction according to step (D) insubstantially, if at all.

During the oxidation in step (D), an acrylic acid comprising gaseous acrylic acid phase arises, whereby acrylic acid is depleted from this acrylic acid phase and at least a part of the depleted acrylic acid phase may be fed into step (A) or (D). Part of the depleted acrylic acid phase before the feeding-in may be subjected to a combustion, such as a gas phase combustion and particularly preferably a catalytic gas phase combustion, as described in WO 03/051809. A depleted acrylic acid phase preferably comprises less than about 5 wt %, or less than about 1 wt %, or less than about 0.1wt % of acrylic acid, respectively based on the depleted acrylic acid phase. Further components of the depleted acid phase may include water, nitrogen, and CO₂. Advantageously, the part of the depleted acrylic acid phase, in particular after the combustion, may be used as carrier gas in the process according to the invention for production of acrylic acid. Furthermore, the oxygen or air flow, respectively necessary for an oxidation of the acrolein, may be introduced either to be used at the same time as carrier gas in step (A) or for the purpose of the oxidation of the acrylic acid directly in step (D).

Furthermore, carbon monoxide may be supplied to the acrolein reaction phase, or if large amounts of carbon monoxide have been formed during the dehydration, that the carbon monoxide may be either selectively oxidized or removed before the bringing into contact with gas phase catalyst, in order to prevent, in particular in the case of metal oxides as gas phase catalyst, a reduction of the catalyst and thus an at least partial inactivation. The carbon monoxide may, for example, be selectively oxidized to carbon dioxide.

The invention further relates to an oxidation device, comprising, connected with each other in fluid-conducting manner,
a dehydration unit;
downstream therefrom, a gas phase oxidation unit;
whereby the dehydration unit comprises
   a reactant feed;
   downstream therefrom, an acrolein reaction area;
   downstream therefrom, a pressure regulator; and
   downstream therefrom, a depletion unit, whereby the depletion unit is connected with the gas phase oxidation unit in fluid-conducting manner;
whereby the gas phase oxidation unit comprises, downstream from the depletion unit
   a reactor, comprising a multioxide catalyst; and
   a processing unit.

The reactant feed may occur by taking the reactant from a tank, which may receive either glycerine as such or glycerine in the form of an aqueous solution. In the context of the acrolein reaction area, reference is first made to the above details. The acrolein reaction area, in the region in which it is formed like a pipe, may have a longer diameter compared to the cross-section.

The pressure regulator following downstream from the acrolein reaction area, from the viewpoint of the reactant feed and in the sense of the flow of reactants and reaction products, may have at least one, or at least two or more pressure regulators, formed as pressure regulating valves—for example as an over-current valve. A depletion unit follows this, in turn, downstream. The depletion unit may directly follow the pressure regulator. This may be used if the depletion of the acrolein from the acrolein reaction phase present before the pressure regulator occurs by release of pressure of the acrolein reaction phase. By these measures, a further reaction of the acrolein phase may be reduced or completely prevented and thus also the formation of undesired side-components.

According to another embodiment of the device according to the invention, the depletion unit may comprise a heat exchanger. This may be provided at the start of the depletion unit. In another embodiment of the device according to the invention, a separation device may follow from the heat exchanger, which is formed as a membrane or crystallizer and in particular as a distillation column. The device according to the invention, either in the acrolein reaction area or before the acrolein reaction area or at both positions, may include a heating element. This heating element may be thermally coupled with the heat exchanger provided in the depletion unit.

The acrolein reaction area may further comprise a dehydration catalyst. This dehydration catalyst may be arranged and fixed in the acrolein reaction area. This may be achieved in that the dehydration catalyst is immobilized at walls of the acrolein reaction area, or, if the dehydration catalyst is present in the form of particles or immobilized thereon, suitable sieves and filters in the acrolein reaction area prevent the flushing-out of these particles.

Furthermore, the oxidation device according to the invention in one embodiment may comprise the multioxide catalyst as powder, layer, or pellet or a combination of at least two thereof. These powders, layers, or pellets may be located at metal walls of metal plates or metal pipes. In the device according to the invention, plate reactors, for example those with thermo plates, or with a plurality of pipes, also called pipe bundle reactors, may be used. In connection with the composition of the multioxide catalysts, reference is made to the details in WO 03/051809 as part of this disclosure, whereby catalysts based on molybdenum, vanadium, and tungsten may be used.

The processing unit may further comprise a quench unit. The device according to the invention may comprise a water separating unit, which is preferably combined with the quench unit and contributes advantageously to the generation of the acrylic acid-depleted acrylic phase, whereby in this context, references are also made to the disclosure of WO 03/051 809.

In a further embodiment of the process according to the invention for production of acrylic acid, this occurs in an above-described device.

The invention also relates to a process for production of a polymer by radical polymerization of the acrylic acid comprising the steps:
i) provision of an optionally partially neutralized acrylic acid and a monomer phase comprising cross-linker, whereby the acrylic acid is obtained according to the above-described process;
ii) radical polymerization of the monomer phase to obtain a hydrogel;
iii) optionally, comminution of the hydrogel;
iv) drying of the hydrogel to obtain a particulate water-absorbing polymer structure;
v) optionally, milling of the particulate water-absorbing polymer structure;
vi) surface post-cross linking of the particulate water-absorbing polymer structure;
vii) bringing into contact of the water-absorbing polymer structure with a coating agent, wherein the bringing-into-contact occurs before, during, or after, particularly preferably after the surface post-cross linking.

This radical polymerization may occur in the presence of cross linkers and using the acrylic acid in at least partially neutralized form, so that in this way cross-linked, water-absorbing polymer structures may be obtained. With respect to the details of the production of such water-absorbing polymer structures based on acrylic acid, reference is made to “Modern Superabsorbent Polymer Technology”, F. L. Buchholz and A. T. Graham, Wiley-VCH-Verlag. The acrylic acid in process step i) may be present to at least about 20 mol %, or to at least about 50 mol %, based on the monomer, as a salt.

With respect to the preferred cross linkers and surface post-cross linking agents, as well as with respect to the amounts and the conditions under which these components are used, as well as with regard to further components which may be present in a monomer solution, as well as with regard to the polymerization conditions, the drying conditions, the comminution, and the surface post-crosslinking, reference is made to DE 103 34 271 A1, whose disclosure limited to cross linkers and surface post-cross linking agents consistent with this invention is hereby incorporated by reference.

As coating agent in process step vii), organic or inorganic materials may be used. As organic material, any optionally particulate organic material known to the skilled person may be used, which is commonly used for modification of properties of water-absorbing polymers. Those organic materials which are mentioned in DE 103 34 286 A1 as fine particulate organic materials belong to the preferred organic materials. Besides these particulate organic materials, those compounds may also be used which are mentioned in WO 02/34384 A1 as nitrogen-containing non-ionic surfactants, or also silicones, as described in EP 0 977 803 A1.

As inorganic material, a particulate, inorganic material known to the skilled person may be used as coating agent, which is generally used to modify the properties of water-absorbing polymers. Those inorganic materials which are mentioned in DE 103 34 286 A1 as fine particulate inorganic materials also belong to the preferred inorganic materials here, whereby zeolites, silicon dioxides, and kaolin are particularly preferred. Further preferred inorganic materials, preferably particulate inorganic materials, are phosphates, as mentioned in WO 02/060983 A2, and aluminum-comprising particles, which are mentioned, for example, in WO 2004/113452 A1, WO 2004/069293 A1, WO 2004/069915 A1, and WO 2005/027986 A1.

The coating agents in process step vii) in an amount of from about 0.01 to about 10 wt %, or in an amount from about 0.1 to about 5 wt %, based on the weight of the water-absorbing polymer structures, may be brought into contact with these structures.

A contribution to the solution of the above-mentioned objects is also made by the water-absorbing polymer structures obtainable by the above described process.

A contribution to the solution of the above-mentioned objects is also made by water-absorbing polymer structures which are based to at least about 25 wt %, or to at least about 50 wt %, or to at least about 75 wt %, or to at least about 95 wt % on acrylic acid, whereby at least about 80 wt %, or at least about 90 wt %, or at least about 95 wt % of the acrylic acid monomers used in the production of the water-absorbing polymer structures have been obtained by the above-described process from glycerine via acrolein as intermediate product, and which have been coated with about 0.01 to about 10 wt. %, based on the weight of the water-absorbing polymer structures, of a coating agent, whereby examples of coating agents are those coating agents that have already been mentioned above in the context of the process according to the invention for the production of water-absorbing polymer structures.

The coating agent may not be a surface post-crosslinker.

According to a particular embodiment of the water-absorbing polymer structures according to the invention, these are based to at least about 25 wt %, or at least about 35 wt %, or at least about 45 wt % on natural, biodegradable polymers, preferably on carbohydrates such as, for example, celluloses or starches.

It is further preferred according to the invention that the water-absorbing polymer structures have at least one of the following properties:
(β1) a CRC value (CRC=Centrifugation Retention Capacity) determined according to ERT 441.2-02 (ERT=Edana Recommended Test Method) of at least about 20 g/g, or at least about 25 g/g, or at least about 30 g/g, whereby a CRC value of about 60 g/g, or of about 50 g/g is not exceeded;
(β2) an absorption under a pressure of 20 g/cm² determined according to ERT 442.2-02 of at least about 16 g/g, or at least about 18 g/g, or at least about 20 g/g, whereby a value of about 50 g/g, or about 40 g/g is not exceeded;
(β3) the polymer structure has a biodegradability determined according to the modified Sturm test according to Appendix V to the Guideline 67/548/EWG after 28 days of at least about 25%, or at least about 35%, or at least about 45%, whereby a value of at most about 75 to about 95% as upper limit is generally not exceeded.

A further contribution to the solution of the above-described objects is provided by a composite comprising the water-absorbing polymer structures according to the invention or respectively water-absorbing polymer structures which may be obtainable by radical polymerization of the acrylic acid obtainable by the above-described process in the presence of crosslinkers. The polymer structures according to the invention and the substrate may be firmly bound to each other. As substrate, sheets made from polymers, such as, for example, from polyethylene, polypropylene or polyamide, metals, non-wovens, fluff, tissues, woven materials, natural, or synthetic fibers, or other foams may be used. The polymer structures may be comprised in an amount of at least about 50 wt %, or at least about 70 wt %, or at least about 90 wt %, based on the total weight of polymer structures and substrate, in the composite.

In a particularly preferred embodiment of the composite according to the invention, it is a sheet-like composite, as described in WO-A-02/056812 as absorbent material. The disclosure of WO-A-02/056812, in particular with respect and limited to the exact construction of the composite, the mass per unit area of its components and its thickness is hereby introduced as reference and represents a part of the disclosure of the present invention.

A further contribution to the solution of the above-mentioned objects is made by a process for production of a composite, whereby the water-absorbing polymer structures according to the invention or respectively the water-absorbing polymers which may be obtainable by radical polymerization of the acrylic acid obtainable by the above-described process in the presence of cross-linkers, and a substrate, and optionally an additive may be brought into contact with each other. As substrate, those substrates may be used that have already been mentioned in connection with the composite according to the invention.

A contribution to the solution of the above-mentioned object may also be made by a composite obtainable according to the above-described process.

A further contribution to the solution of the above-mentioned objects may be made by chemical products comprising the water-absorbing polymer structures according to the invention or a composite according to the invention, or based on the acrylic acid obtainable by the process according to the invention. Examples of chemical products may include fibers, sheets, formed masses, textile and leather additives, flocculants, coatings, varnishes, foams, films, cables, sealant materials, liquid-absorbing hygiene articles, in particular diapers and sanitary napkins, carriers for plant or fungus growth-regulating agents or plant protection active agents, additives for construction material, packaging materials, or soil additives.

Hygiene articles according to the invention may comprise a top sheet, a bottom sheet, and an intermediate sheet arranged between the top sheet and the bottom sheet, which may comprise the water-absorbing polymer structures according to the invention.

The use of the water-absorbing polymer structures according to the invention or of the composite according to the invention in chemical products, in the above-mentioned chemical products, in particular in hygiene articles such as diapers or sanitary napkins, as well as the use of the water-absorbing polymer structures as carrier for plant or fungus growth-regulating agents or plant protection active materials also make a contribution to the solution of the above-mentioned objects. In the use as carrier for plant or fungus growth-regulating agents or plant protection active substances, it is preferred that the plant or fungus growth-regulating agents or plant protection active substances may be released over a time period controlled by the carrier.

The present invention is now more closely described by means of non-limiting diagrams and examples.

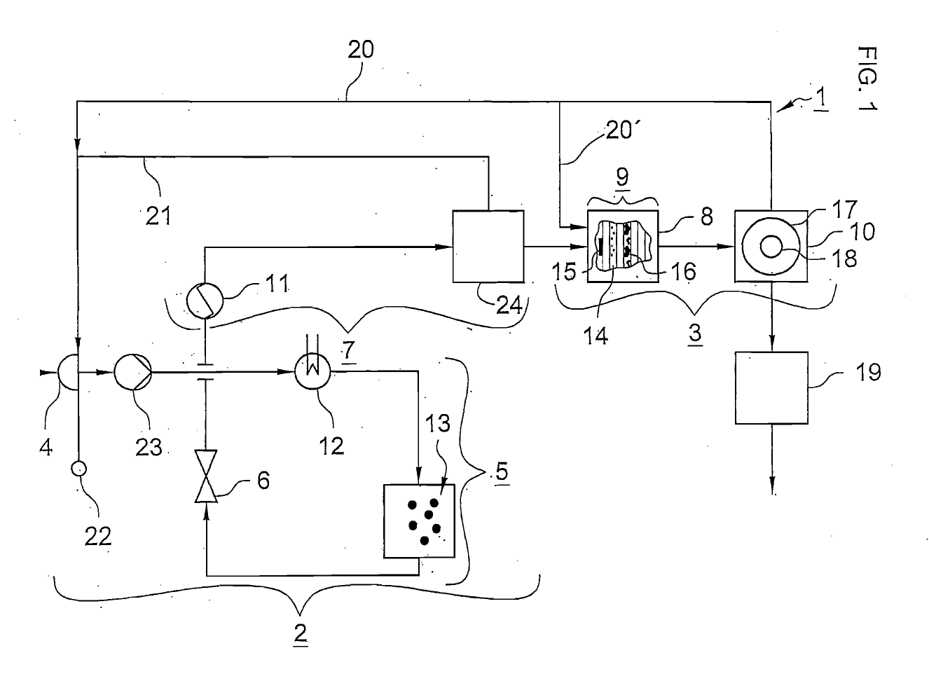

FIG. 1 shows schematically a device 1 according to the invention for dehydration and oxidation, comprising a dehydration unit 2, which is connected with a gas phase oxidation unit 3 in fluid-conducting fashion, i.e. connected flow-technologically with each other in such a way that both liquid and gas may be conducted. The dehydration unit 2 receives, via a reactant feed 4, glycerine or respectively an aqueous solution of glycerine, which may be pre-stored in a tank which is not shown. By means of a pressure generator 23 designed as a high pressure pump (for example a multipiston pump from the company Lewa, Germany) the aqueous glycerine in an acrolein reaction area 5 (such as a stainless steel pipe) is compressed against a pressure regulator 6 (for example formed as over-current valve) and, if necessary, further heated by means of a heating element 12. The acrolein reaction area 5 may further comprise a dehydration catalyst 13 immobilized therein, or liquid catalyst may be supplied, at which the glycerine reacts to form acrolein. By means of the pressure regulator 6, the thus-formed acrolein is discharged from the acrolein reaction area 5 which is under high pressure by release of pressure into a depletion unit 7. The depletion unit 7 may in turn comprise a heat exchanger 11, which is thermally coupled with the heating element 12. In the depletion unit 7, a distillation device 24 may follow from the heat exchanger 11 usable for the cooling. An acrolein-poor acrolein reaction phase leaves the depletion area 7 and in particular the distillation device 24 via a back-conduit 21, in order to be supplied via reactant feed 4 to the acrolein reaction area 5, in order to conduct the glycerine still present in the acrolein-poor acrolein reaction phase to a further dehydration. Furthermore, an acrolein-rich acrolein phase leaves the depletion unit 7 into the gas phase oxidation unit 3 following the depletion unit 7. The gas phase oxidation unit 3 comprises, in turn, a reactor 9, which comprises, in pipe walls represented schematically as pipe cross-section, catalyst powder 14 or a catalyst layer 15 or catalyst pellets 16. A processing unit 10 follows the reactor 9. This processing unit comprises a quench unit 17 formed as a quench column and a water separating unit 18. From the processing unit 10, via a back-line 20 or 20′ respectively, an acrylic acid-poor acrylic acid phase may be supplied to the reactant feed 4 or respectively to the reactor 9. An acrylic acid-rich acrylic acid phase is supplied from the processing unit 10 to a purification unit 19, which is, for example, designed as crystallization unit, as described in DE 102 11 686. The acrylic acid obtained here from in high purity may, furthermore, be further processed to polyacrylates and in particular also as water-absorbing polymers characterized as superabsorbers.

### EXAMPLE 1

A glycerine solution (5 wt. % in water, acidified with phosphoric acid in the ratio 1:2000, based on the glycerine) was supplied at 360 ml/h into a reactor (acrolein reaction area 5) with a volume of 95 ml. The pressure in the reactor was maintained at 150 bar. The reactor was brought to a temperature with a maximum of 345° C. by means of secondary heating. The turnover in first throughput was 89.6%, the selectivity for acrolein was 80.2%, and the yield of acrolein in the first throughput was 71.8%. The phase from which acrolein was removed was conducted back into the reactor for simulation of a continuous circuit.

### EXAMPLE 2

A glycerine solution (5 wt. % in water, acidified with phosphoric acid in the ratio 1:2000, based on the glycerine) was fed at 480 ml/h into a reactor with a volume of 95 ml. The pressure in the reactor was maintained at 150 bar. The reactor was brought to a temperature with a maximum of 345° C. by means of secondary heating. The turnover was 29.5%, and the selectivity for acrolein was 73.7%.

### EXAMPLE 3

The hot product stream at 180-220° C. in the form of vapor from the dehydration reactor, with a composition of 15 wt. % acrolein, 82 wt. % water vapor, and the remainder other lower boiling components was, analogously to WO 03/051809 A1, together with 1.5 kg/h pre-heated air, fed into an oxidation reactor which is filled with 1.8 1 commercial V-Mo multioxide catalyst.

The acrolein/water vapor/air mixture from the dehydration reactor was converted at 250° C. and slightly increased ambient pressure with a GHSV of 280 Nl acrolein/(1 cat·h) and in the reactant mixture, with an acrolein turnover of 99.5 mol %, an acrylic acid yield of 93 mol % was obtained.

### EXAMPLE 4

A monomer solution consisting of 280 g of the above obtained acrylic acid, which was neutralized to 70 mol % with sodium hydroxide, 466.8 g water, 1.4 g polyethylene glycol-300-diacrylate, and 1.68 g allyloxypolyethylene glycol acrylic acid ester was purged with nitrogen to remove dissolved oxygen and cooled to a starting temperature of 4° C. After reaching the starting temperature, the initiator solution (0.1 g 2,2′-azobis-2-amidinopropane dihydrochloride in 10 g H₂O, 0.3 g sodium peroxydisulfate in 10 g H₂O, 0.07 g 30% hydrogen peroxide solution in 1 g H₂O, and 0.015 g ascorbic acid in 2 g H₂O) was added. After the end temperature of approximately 100° C. was reached, the resulting gel was comminuted and dried for 90 minutes at 150° C. The dried polymer was coarsely chopped, milled, and sieved to a powder with a particle size from 150 to 850 μm.

For the cross-linking, 100 g of the above-obtained powder was mixed with vigorous stirring with a solution of 1 g 1,3-dioxolan-2-one, 3 g water and 0.5 g aluminum sulphate-18-hydrate, and then heated for 40 minutes in an oven which was regulated to 180° C.

After cooling, the water-absorbing polymer particles are sprayed with a 50% aqueous slurry of Kaolin (NeoGen, DGH®) in such an amount that the water-absorbing polymer structure was coated with 3 wt. % Kaolin.

### EXAMPLE 5

### Preparation of a Biodegradable Polymer

The post-crosslinked polymer surface-treated with kaolin obtained in Example 4 was mixed under dry conditions with a water-soluble wheat starch (the product Foralys® from the company Roquette, Lestrem, France) in the weight ratio polymer:starch of 4:1 and then further homogenized for 45 minutes in a roll mixer type BTR 10 from the company Fröbel GmbH, Germany.

### LIST OF REFERENCE NUMERALS

1 oxidation device
2 dehydration unit
3 gas phase oxidation unit
4 reactant feed
5 acrolein reaction area
6 pressure regulator
7 depletion unit
8 reactor
9 multioxide catalyst
10 processing unit
11 heat exchanger
12 heating element
13 dehydration catalyst
14 powder
15 layer
16 pellet
17 quench unit
18 water separation unit
19 purification unit
20, 20′ conducting back of the acrylic acid-poor acrylic acid phase
21 conducting back of the acrolein-poor acrolein reaction phase
22 CO-feed
23 pressure generator
24 distillation device

1. A process for production of acrolein comprising the following steps:
(a) bringing an aqueous glycerine phase into an acrolein reaction area to obtain an aqueous acrolein reaction phase wherein the acrolein reaction phase is at least partially in the supercritical area;
(b) depleting the acrolein from the acrolein reaction phase to obtain an acrolein phase and a depleted acrolein reaction phase; and
(c) conducting back at least a part of the depleted acrolein reaction phase into the acrolein reaction area.

2. A process for production of acrolein having the following steps:
(a) bringing an aqueous glycerine phase into an acrolein reaction area to obtain an aqueous acrolein reaction phase, wherein the acrolein reaction phase in the acrolein reaction area has a pressure of at least about 80 bar and a temperature of at least about 320° C.;
(b) depleting the acrolein from the acrolein reaction phase to obtain an acrolein phase and a depleted acrolein reaction phase; and
(c) conducting back at least a part of the depleted acrolein reaction phase into the acrolein reaction area.

3. A process for production of acrylic acid, comprising the following steps:
(A) bringing an aqueous glycerine phase into an acrolein reaction area to obtain an aqueous acrolein reaction phase;
(B) depleting the acrolein from the acrolein reaction phase to obtain an acrolein phase and a depleted acrolein reaction phase;
(C) conducting back at least a part of the depleted acrolein reaction phase into the acrolein reaction area; and
(D) oxidizing the acrolein from the acrolein phase to acrylic acid in the gas phase at a gas phase catalyst.

4. The process according to claim 3, wherein the acrolein reaction phase in the acrolein reaction area has a pressure of at least about 50 bar.

5. The process according to claim 3, wherein the acrolein reaction phase in the acrolein reaction area has a temperature of at least about 100° C.

6. The process according to claim 3, wherein the acrolein reaction area comprises a dehydration catalyst.

7. The process according to claim 6, wherein the dehydration catalyst is an acid or a base.

8. The process according to claim 7, wherein the acid is an inorganic acid.

9. The process according to claim 7, wherein the acid is an organic acid.

10. The process according to claim 3, wherein the acrolein reaction phase comprises a liquid different from water.

11. The process according to claim 10, wherein the liquid different from water is aprotic and polar.

12. The process according to claim 3, wherein the acrolein reaction area comprises a metal or a metal compound or both.

13. The process according to claim 3, wherein the residence time of the acrolein reaction phase is from about 1 to about 10000 seconds.

14. The process according to claim 3, wherein the acrolein phase comprises carbon monoxide.

15. The process according to claim 3, wherein the glycerine phase comprises less than about 10 wt % glycerine.

16. The process according to claim 3, wherein the turnover in the acrolein reaction phase is at least about 25%.

17. The process according to claim 3, wherein the acrolein reaction phase at the end of the acrolein reaction area comprises an amount of less than about 50 wt. % glycerin, based on the acrolein reaction phase.

18. The process according to claim 3, wherein the acrolein reaction phase at the end of the acrolein reaction area comprises an amount within the range from about 0.1 to about 50 wt. % of acrolein, based on the acrolein reaction phase.

19. The process according to claim 3, wherein at least a part of the acrolein reaction phase is gaseous.

20. The process according to claim 3, wherein the acrolein reaction phase in the acrolein reaction area is present in at least two aggregate states.

21. The process according to claim 3, wherein the acrolein reaction phase before the depletion is under higher pressure than during the depletion.

22. The process according to claim 3, wherein the acrolein in the acrolein reaction area is at least partially present in the supercritical state.

23. The process according to claim 3, wherein the acrolein concentration in the acrolein reaction phase before the depletion is at least about 5% higher than after the depletion.

24. The process according to claim 3, wherein a carrier gas is used.

25. The process according to claim 24, wherein the carrier gas is at least partially fed back into the acrolein reaction area after passing through the acrolein reaction area.

26. The process according to claim 3, wherein the acrolein phase in step (D) comprises acrolein within a range from about 5 to about 30 wt. %, based on the acrolein phase.

27. The process according to claim 3, wherein during the oxidation an acrylic acid-comprising gaseous acrylic acid phase forms, wherein acrylic acid is depleted from this acrylic acid phase and at least a part of the depleted acrylic acid phase is fed into step (A), or (D), or both.

28. A device for dehydration and oxidation, connected with each other in fluid-conducting manner, comprising
a dehydration unit;
downstream therefrom a gas phase oxidation unit;
wherein the dehydration unit comprises
a reactant feed;
downstream therefrom an acrolein reaction area;
downstream therefrom a pressure regulator; and
downstream therefrom a depletion unit,
wherein the depletion unit is connected in fluid-connecting manner with the gas phase oxidation unit;
wherein the gas phase oxidation unit comprises, downstream from the depletion unit
a reactor, comprising a multioxide catalyst; and
a processing unit.

29. The device according to claim 28, wherein the depletion unit comprises a heat exchange.

30. The device according to claim 28, wherein the acrolein reaction area can be heated by means of a heating element.

31. The device according to claim 28, wherein the acrolein reaction area comprises a dehydration catalyst.

32. The device according to claim 28 wherein the dehydration catalyst is immobilized in the acrolein reaction area,

33. The device according to claim 28, wherein the multioxide catalyst is present as powder, layer or pellet or a combination of at least two therefrom.

34. The device according to claim 28, wherein the processing unit comprises a quench unit.

35. The device according to claim 28, wherein the processing unit comprises a water separation unit.

36. (canceled)

37. A process for production of water-absorbing polymer structures, comprising the process steps:
i. provision of an optionally partially neutralized acrylic acid and a monomer phase comprising crosslinker, wherein the acrylic acid is obtained according to a process according to claim 3;
ii. radical polymerization of the monomer phase to obtain a hydrogel;
iii. optionally, comminution of the hydrogel;
iv. drying the hydrogel to obtain a particulate water-absorbing polymer structure;
v. optionally, milling of the particulate water-absorbing polymer structure;
vi. surface post-crosslinking of the particulate water-absorbing polymer structure; and
vii. bringing into contact of the water-absorbing polymer structure with a coating agent, wherein the bringing into contact occurs before, during or after the surface post-crosslinking.

38. The process according to claim 37, wherein the acrylic acid is present to at least about 20 mol % based on the monomer, as a salt.

39. Water-absorbing polymer structures, obtainable by a process according to claim 37.

40. A water-absorbing polymer structure, which is based to at least about 25 wt. % on acrylic acid, wherein at least about 80 wt. % of the acrylic acid monomer used in the production of the water-absorbing polymer structures, has been obtained by the process according to claim 3, and which is coated with from about 0.01 to about 10 wt. %, based on the weight of the water-absorbing polymer structures.

41. The water-absorbing polymer structure according to claim 40, wherein the polymer structure is based to at least about 25 wt. %, based on the total weight of the water-absorbing polymer structures, on natural, biodegradable polymers.

42. A composite including a water-absorbing polymer structure according to claim 39 and a substrate.

43. A process for production of a composite according to claim 42, wherein the water-absorbing polymer structure and the substrate are brought into contact with each other.

44. A composite obtainable by a process according to claim 43.

45. A hygiene article, comprising a top sheet, a bottom sheet and an intermediate sheet, arranged between the top sheet and the bottom sheet, which includes water-absorbing polymer structures according to claim 39.

46. Fibers, sheets, formed masses, textile and leather additives, flocculants, coatings, or varnishes based on acrylic acid obtainable according to a process according to claim 3 or 36 or derivatives, or salts thereof.

47. Use of an acrylic acid obtainable according to a process according to claim 3 or derivatives, or salts thereof in fibers, sheets, formed masses, textile, and leather additives, flocculants, coatings, or varnishes.
